(19) United States
(12) Patent Application Publication
(10) Pub. No.: US 2008/0248050 A1
(43) Pub. Date: Oct. 9, 2008

(54) Meta-specific vaccine, method for treating patients immunized with meta-specific vaccine

(75) Inventor: Fred J. Stevens, Naperville, IL (US)

Correspondence Address: CHERSKOV & FLAYNIK, THE CIVIC OPERA BUILDING, 20 NORTH WACKER DRIVE, SUITE 1447, CHICAGO, IL, 60606, US

(73) Assignee: UChicago Argonne, LLC

(21) Appl. No.: 11/824,726

(22) Filed: Jul. 2, 2007

Related U.S. Application Data
(60) Provisional application No. 60/817,940, filed on Jun. 30, 2006.

(51) Int. Cl. A61K 39/395; C07K 16/18; A61P 37/00

(52) U.S. Cl. 424/178.1; 530/387.1; 530/391.1

(57) ABSTRACT

A meta-specific vaccine particle is provided. Also provided is a method for inducing an immune response in a human (homo sapiens) or non-human previously exposed to the meta-specific vaccine, and subsequently infected with a pathogen.

This utility application claims the benefits of U.S. Provisional Application No. 60/817,940 filed on Jun. 30, 2006.

### CONTRACTUAL ORIGIN OF THE INVENTION

The United States Government has rights in this invention pursuant to Contract No. W-31-109-ENG-38 between The University of Chicago and Argonne National Laboratory.

### BACKGROUND OF THE INVENTION

1. Field of the Invention

This invention relates to a universal vaccine, and more particularly, this invention relates to systematic modification of a library of phage particles having the commutative effect of shutting down heretofore incorrigible biological systems.

2. Background of the Invention

There is a growing concern about the risk of pandemic, whether originating from novel bioengineered pathogens or from newly emergent diseases; e.g., “bird flu”. Complicating the issue is the increasing resistance of bacteria to antibiotics, a feature that would likely be incorporated into a terrorist-engineered pathogen. Also, viruses are not vulnerable to antibiotics.

Traditional vaccines require tremendous effort to produce in large quantities, even when the vaccine target is known and available for study. Production of a vaccine against a new target first requires enabling research and development to identify the specific targets and develop methods for vaccine development. Using traditional approaches, a vaccine against a bioengineered pathogen or a new, naturally emerging disease would not be available in significant quantities for at least two years, assuming that the FDA and other government agencies waive the clinical trials which are normally required to establish safety and efficacy.

The generation of knowledge that enables the efficient stabilization of antibodies will allow the full potential of phage display technology to be realized. In phage display technology, genetic material that encodes human or mouse immune components are added to the DNA of phage (viruses that infect bacteria). Each phage has a marker, comprising variable domain components, that is displayed on the exterior or outside surface of the phage, thereby forming a phage-marker construct.

Hundreds of the aforementioned constructs are arranged to produce libraries of variable domain components. When these libraries are exposed to a target of interest, such as viruses including HIV and bird flu, those phage that display an scFv construct that recognizes a protein on the target, bind to it. Thus, when phage that do not bind are removed, what is left is a collection of bacterial viruses that contain human or mouse DNA that encodes an antibody capable of interacting with that target of interest. That DNA can then be transferred to E. coli or yeast with the expectation that large quantities of protein can be produced for use. However, in many cases, little or no protein is obtained. Often when soluble protein is obtained, it rapidly precipitates or requires special handling. Even an scFv of better than average stability must be handled with care.

Efforts have been made to stabilize Fabs and scFvs. For example, Demerast et al, Protein Engineering, Design and Selection, 19, pp 325-336 (2006) compiled a database of immunoglobulin sequences and calculated entropy to identify residue positions having the highest degree of variability. While the reasoning there was these variable positions would be tolerant of substitution, a larger proportion of the positions are actually destabilizing. It was only through several hundred substitutions that stabilizing positions were identified.

The aforementioned limitations associated with unstable antibodies, and the time consuming analysis required to identify stable residue positions have stymied antibody application in medical and nonmedical endeavors.

This unpredictability has blocked many potential projects and has prompted many groups to abandon antibodies and to attempt to create libraries based on other protein “scaffolds”. However, the drawback to abandoning antibody research includes abandoning the diversity that is naturally embedded in antibody libraries. By retaining that diversity, the possibility of a “universal vaccine” may exist.

HIV presents an unprecedented challenge for vaccine development. It is well known that this virus has a high rate of mutation in its reproduction, a fact that limits the effectiveness of drugs developed to inhibit enzymes the virus depends upon for its replication as well as the outer surface that would be the target of the immune response evoked by a vaccine. In the case of HIV, a conventional vaccine would stimulate an immune response capable of recognizing only part of the population of HIVs present in an infectious encounter and would not recognize many of the viruses developed during the course of infection.

A need exists in the art for a protocol to produce stable antibody subunits. The protocol, utilizing single chain variable fragments of well characterized human antibodies, would allow rapid scale-up of production of scFVs within days after identification of a new bacterium or virus.

### SUMMARY OF THE INVENTION

An object of the present invention is to produce a universal vaccine that overcomes many of the disadvantages of the prior art.

Another object of the present invention is to enable the production of pathogen fighting substances and medicaments. A feature of the invention is the stabilization of components of well-characterized antibodies. An advantage of the invention is that the stabilized components can enable large-scale production of the pathogen-fighting substances and medicaments in short notice.

Still another object of the present invention is the creation of a unique structural determinant. A feature of the invention is an antibody component, such as a mouse or human single chain variable fragment that is modified by the addition of a predetermined moiety. An advantage of this invention is that immunization of individuals with the structural determinant will result in an immune response to the determinant. Another advantage is that the remainder of the human scFv is invisible to the human immune system.

Yet another object of the present invention is a method for developing and storing single chain variable fragments. A feature of the method is that it will enable the existence of robust scFvs, thereby allowing large scale production of the scFv to be achieved within a few days after the identification of a threatening new bacterium or virus. An advantage of the method is that safety and efficacy testing of a newly developed scFv can be achieved with surrogate scFvs inasmuch as only a few amino acids will distinguish one scFv from another.

Still another object of the present invention is a method for stockpiling medicaments and like materials to control known threats. Another object is to provide public health officials with a strategy and means to respond to newly emerging challenges. A feature of the method is using basic building blocks of existing antibody families and slightly modifying these building blocks. An advantage of the method is the production of a knowledge-base of amino acid variations to enable assured robustness of all antibodies to be used in new biotechnological applications ranging from immunodiagnostics to detection of explosives.

Briefly, the invention provides for a substance for detecting a target molecule, the substance comprising a first antibody molecule fragment; and a second molecule in close spatial relationship to the fragment; the second molecule comprising a means to induce an immune response

Also provided is a method for inducing a universal immune response in a patient comprising obtaining human scFv comprised of a variable light chain and a variable heavy chain, whereby the light chain and heavy chain are connected by a peptide and wherein the scFv has meta-specificity, modifying the peptide chain by attaching a compound to the chain, obtaining protein coats for a pathogen, isolating and immobilizing pathogen coat proteins, identifying antibody components which interact with the pathogen; infecting bacteria with phage containing surface modifiers which interacted with VX proteins in step e, generating homogeneous populations of the phage, stabilizing DNA contained in the phage that expresses pathogen specific scFv, introducing the pathogen specific scFv DNA into bacteria or yeast to induce production of protein, producing and purifying the protein, modifying the protein by attachment of the same compound heretofore connected to the meta-specific scFv, and infusing patients with the modified protein, those patients previously immunized with scFV-alpha.

The invention further provides for a universal antibody produced by the following process: isolating a scFv component having meta-specificity; producing quantities of the component; and attaching a moiety to the produced scFv component quantities, whereby the moiety induces an immune response.

### BRIEF DESCRIPTION OF THE DRAWING

The foregoing and other objects, aspects and advantages of this invention will be better understood from the following detailed description of the preferred embodiments of the invention with reference to the drawing, in which:

FIG. 1 is a schematic diagram for a process for producing stabilized scFv, in accordance with features of the present invention;

FIG. 2 is a schematic diagram of an antibody component modified by a determinant molecule, in accordance with features of the present invention;

FIG. 3 is a graph of mutated scFv bound to laminin-1, in accordance with features of the present invention; and

FIG. 4. is a table showing various mutations of scFv and their binding characteristics to laminin, in accordance with features of the present invention.

### DETAILED DESCRIPTION OF THE INVENTION

Mouse and human antibodies are used for virtually all biotechnological applications of antibodies. These are produced by combinations of a roster of approximately 50 genes each, in which genetic substitutions create different binding specificities, often at the price of substantial penalties in stability. Although the 50 gene starting points are similar in sequence to each other, they also have significant inherited variations. Some of those variations are intrinsically destabilizing, while others are intrinsically stabilizing.

Suitable antibodies and their components known for stabilization qualities are derived from human and nonhuman (e.g., mouse) antibodies. The universal vaccine derived from the invented stabilized antibody components will require human antibodies. For other applications, such as in sensors, chemical purification processes, remediation, “smart” tape (i.e., material that sticks to itself but nothing else) and others, if the antibody can be selected out of a library, either human or mouse antibodies are available. Traditional monoclonal antibodies generally are derived from mice. Whether obtained from immunization or from a library, and whether human or mouse, stabilization of antibodies by stabilization of its components is possible in every case.

An embodiment of the invention is systematic identification of those amino acid changes that improve stability by using known sequences to guide site-specific mutational analyses by well developed methods. The key concept for systematically stabilizing antibodies and, hence, enabling a universal vaccine and other applications is that genomic data provides a menu of potentially stabilizing amino acid changes.

Stability can be measured in several ways. In general, stability is quantified as either thermodynamic stability or thermal stability. The two measurements are correlated but are not exactly the same. Thermodynamic stability is quantified in terms of a denaturant concentration that yields an equilibrium between folded and unfolded forms of the protein. Such quantification is found in Stevens et al. Protein Science 4: 421-432 (1995) and incorporated herein by reference.

Thermal stability is related to a temperature at which half the protein is unfolded (in a defined period of time).

The invented process of enhancing the stabilization of antibody fragments greatly enhances phage display libraries, which in turn provide the most rapid means for generating new antibodies against newly identified pathogens.

A myriad of well-known mutations exist in the art and are suitable in stabilizing antibodies and antibody fragments of interest. As such, the site specific mutations listed below are for illustrative purposes only. Further, while human kappa-1b is featured in this stability illustration:
asp 1
ile
gin
met
thr
gin
ser
pro
ser
ser 10
leu
ser
ala
ser
val
gly
asp
arg
val
thr 20
ile
thr
cys
gin
ala
ser
gin
asp 27a
ile 27b
gap 27c
gap 27d
gap 27e
gap 27f
gap
gap
ser 30
asn
tyr
leu
asn
trp
tyr
gin
gin
lys
pro 40
gly
iys
ala
pro
lys
leu
leu
ile
tyr
asp 50
ala
ser
asn
leu
glu
thr
gly
val
pro
ser 60
arg
phe
ser
gly
ser 65
gly
gap 66a
gap 66b
ser
gly
thr
asp 70
phe
thr
phe
thr
ile
ser
ser
leu
gin
pro 80
glu
asp
ile
ala
thr
tyr
tyr
cys
gin
gin 90
tyr
asp
asn
leu
pro 95
gap 95a
gap 95b
gap 95c
gap 95d
---
---
---
---
--- 100
---
---
---
---
---
---
gap 106a
--- 107
   (germlne O18-08, SEQ. ID. No. 1,), other antibodies, both human and nonhuman, are suitable stabilization candidates. This sequence is also found in Klein et al., Eur J. Immunol 23, 3248-3271 (1993) and incorporated herein. The strand represented by SEQ. ID. No. 1 is presented in a standard template, which includes the germline-encoded V domain and a short, separate J-segment (joining segment). O18-08 is the germline V gene and does not contain a J-segment. Therefore, the positions indicated by “---” are not part of O18-08 and can be ignored.

Many more stabilizing amino acid substitutions remain to be identified and it is expected that the same substitution may have different consequences in antibodies formed by different gene products.

Table 1 below provides a spectrum of relative stability that single point mutations confer to the kappa protein. Cm indicates molar concentration of denaturant at which half the protein molecules are unfolded. “Base” indicates the stability of the starting variable domain (human kappa-1b). Taking the first mutant Q37L as an example, Q=original glutamine, 37=position of the mutant on the kappa 1-b, and L=leucine inserted to replace glutamine. Numbers larger than base indicate replacements that improve stability. Combination of the last four stabilizing variations shown in Table 1 improves stability by a factor of 2000, i.e., increased the Cm the most.

The inventors found that the stabilizing effects of the mutations they imposed on the genome are additive inasmuch as the amino acid changes are scattered throughout the sequence structure. Thus, the new amino acid side chains do not compete with each other because they are remotely (either distance-wise or electronically) positioned relative to each other. For example, because of their remote placement on the genome, the new side chains do not make hydrogen bonds with the same atom or try to occupy the same space in the molecule. In some cases, changes at adjacent positions are more additive than positions separated by one or a plurality of amino acids, given that the side chains point in different directions along the peptide string.

TABLE 1
Effect of mutant presence on stability of antibody

| Mutant | Cm |
|---|---|
| Q37L | 0.73 |
| I21L | 1.06 |
| R18P | 1.07 |
| A13L | 1.20 |
| V58I | 1.29 |
| base | 1.33 |
| L78I | 1.33 |
| L11V | 1.35 |
| L47I | 1.47 |
| A13V | 1.52 |
| F73L | 1.66 |
| L78V | 1.74 |

Various combinations of stabilizing variations in single molecules verify that the improvements are additive. A natural result of the endeavor is an “engineering almanac.” On the basis of information in this knowledge base, any antibody developed for biotechnological applications can be substantially stabilized at the early stages of a project, without sacrifice of binding properties. Improved stability will shorten the period of research and development, will reduce the cost of production, and will extend shelf life of the product. Of major importance, the invented protocol enables a broad range of novel antibody-based detection systems. In general, improved stability of antibodies will significantly enable the “technology” component of biotechnology, which will be less restricted to biomedical applications.

Below is an example of a sequence (SEQ. ID. No. 2) of one of numerous scFv constructs that exist. Appropriate amino acids are substituted in the linker between the heavy and light chain variable domains: a lysine replaces a glycine at position 122 in this example. Other amino acids are also suitable, depending on the chemistry of attaching the hapten or moiety known to induce immune response. This example of lysine replacing glycine at position 122 is merely illustrative and should not be construed as limiting the invented stabilized constructs. Rather, this example is provided to illustrate that it is possible to introduce chemically modifiable residues and expose same on the peptide linker, without impairing the stability or the scFv. The “foreign” residue on the linker has no adverse effect on the stability of the scFv inasmuch as it does not participate in determining the structure of the variable domains.

```
(SEQ. ID. No. 2)
    1 maaqiqlvqs gpelkkpget veisckasgy tftdygmnwm kqapgkslkw mgwintytge
  61 ptyadefkgr fafsletsas tayiqinnlk sedmatyfcs rsmkgsywgq gtlvtvsagg
121 ggsggggsgg ggsdvvmtqt pltlsvtigq pasisckssq sllgsdgktf lnwllqrpgq
181 spkrliylvs kldsgvpdrf tgsgsgtdft lkisrveaed lgvyycwqgt hlpqtfgggt
241 kleik
(SEQ. ID. No. 3)
    1 maaqiqlvqs gpelkkpget veisckasgy tftdygmnwm kqapgkslkw mgwintytge
  61 ptyadefkgr fafsletsas taylqinnlk sedmatyfcs rsmkgsywgq gtlvtvsagg
121 gKsggggsgg ggsdvvmtqt pltlsvtigq pasisckssq sllgsdgktf lnwllqrpgq
181 spkrliylvs kldsgvpdrf tgsgsgtdft lkisrveaed lgvyycwqgt hlpqtfgggt
241 kleik
```

With the advent of stable antibodies and components of antibodies, several new markets are envisioned, including:
A broad range of diagnostic applications in such “front-line” endeavors involving the military, first responders, and veterinary medicine in which “real-time” diagnostics in the field may be advantageous.
Agricultural diagnostic tests for evaluation of diseased plants or to test for the presence of parasites in water and soil samples.
Improved therapeutic applications, both in the growing field of immuno-therapeutics finding current applications in treating some cancers as well as rheumatoid arthritis and possible future application for treatment of infection by antibiotic-resistant bacteria. Novel therapeutic strategies for removal of toxins from the blood stream are based on coupling antibodies to nano-scale magnetic particles.
Molecularly-specific purification methods for chemical synthesis processes.
Environmental cleanup to remove contaminants ranging from chemicals to anthrax spores to radionuclides.
Novel biosensors to detect explosives, gases, as well as aerosolized toxins, viruses, bacteria and spores.
Possible universal vaccine.

The basic strategy to be used to stabilize antibodies is not restricted to this class of protein. It is expected that most proteins of biotechnological interest can be made robust in this manner, opening other market opportunities.

The present invention is a generic human single chain variable fragment that has been modified at a predetermined site along the fragment. Replacements are selected from the public domain data describing the sequences of the germline genes of the variable domains. The kappa germlne genes are found in Klein et al, discussed and incorporated be reference above. Another public source for germlne sequences (specifically of the lambda variable domains) is Kawasaki et al. Genome Res. 7, 250-261 (1997) and incorporated herein by reference.

All variable domain sequences, including light and heavy, are publically available through the National Center for Biotechnology Information, the National Library of Medicine, National Institutes of Health, Bethesda, Md., and also through its website at http://www.ncbi.nim.nih.gov/igblast/showGermline.cgi. This data informs drug designers using the invented protocol which variations, found within a germline, to insert into the immunoglobulin of interest. Among the variations found in a germlne gene is a high percentage of replacements that improve the stability of the immunoglobulin of interest. When designers are working on a domain that originated from germline A, and all of its accumulated somatic mutations that compromised stability, some of the alternative amino acids that distinguish germline genes B, C, and D will stabilize the antibody descended from A. They will also usually stabilize any antibody descended from A. Inasmuch as there are also replacements from B, C, and D germlines that destabilize the protein, this information steers designers away from utilizing those destabilizing variations observed in those alternative germlines. An example of different strands of the same germline having different amino acid residues at a specific location is position 27 on SEQ. ID. No. 1.

The modification comprises adding a molecule to the fragment so as to create a unique structural determinant. Examples and functions of such modifying molecules are found in D. R. Livesay et al., Protein Engineering, Design and Selection 17, No. 5, pp 463-472 (2004), the entire paper of which is incorporated herein by reference. As such, the molecule could be a marker or complementary genetic sequence for a compound such as tissue specific endothelial cell markers (i.e., laminin), antigens or pathogens generally. The modified fragment would not be on the phage. Modification would take place after the gene had been transferred to E. coli or other organism for production. Purified scFv would be chemically modified.

In the case of HIV, instead of developing a comprehensive vaccine based on the virus itself, phage display technology is used to construct a library that contains components capable of interacting with large numbers of HIV components, sufficient to assure attachment to phage. However scFv, by definition do not have the molecular constituents that enable them to interact with immune cells that would eliminate the virus. Per the invented protocol, the modifier is that recognized by the antibodies which were previously evoked by vaccination with the modifier.

The universal vaccine is a generic human scFv, that has been modified at an appropriate site by addition of an appropriate small molecule. Suitable small molecules include, but are not limited to haptens, inorganic compounds, organic compounds, antibody fragments, semi-conductor particles such as titanium dioxide, metals, and synthetic materials. This creates a unique structural determinant. Immunization of individuals with this “vaccine” will result in an immune response to this determinant, while the remainder of the human scFv is invisible to the human immune system. The scFv proteins in the anti-HIV library would carry the same modification.

HIV patients are immunized with the universal vaccine upon diagnosis, or may have been pre-immunized on the basis of potential accidental exposure or choice. In either case, injection of a diverse scFv cocktail will result in binding to HIV particles and will evoke a response by the immune system. This “cocktail” would include a mixture of several scFv constructs that interact with different HIV markers so that even in the background of mutation, several are likely to bind. All of the scFv constructs will have been combined with the same modifier.

Maintenance of a therapeutic dosage of the scFv may result in disease remission or at least control.

The utility of the universal vaccine strategy is not limited to HIV treatment, which may represent the most difficult challenge. In principle, this approach allows recruitment of the human immune system to any disease state for which a distinctive immunological marker exists. This situation is always the case for viral, bacterial, and parasitic infections. It is also the case for at least some cancers. For instance, multiple myeloma is a cancer involving cells that produce antibodies. A form of the particular antibody is present on the surface of the cell. Its distinctive structural features are unique to each patient. However, the progression of the disease is usually fairly slow. In this instance, efficient phage display technology and stabilization is completed within weeks at which point the patient can be treated with a drug, developed for one patient, that can direct the power of the patients own immune system against cancerous cells through the use of the universal vaccine. By extension, the universal vaccine strategy can offer protection against diseases that have yet to emerge or diseases that re-emerge via acquired antibiotic resistance.

The feasibility of achieving the stated goal is demonstrated by work in our laboratory that has increased the stability of the protein produced by one human antibody gene by a factor of 100,000. This means that the proportion of protein that is “unfolded” at any given instant was decreased by a factor of 100,000. Since it is the unfolded population that is prone to precipitation, to a first approximation the rate of decay of this molecule was decreased by a factor of 100,000 resulting in a predicted rate that is negligible.

For example, the inventors have conferred stability to human kappa4 domain via the following substitutions:
M4L (methionine at position 4 replaced by leucine)
A19V
Y27bL
Y27dD
S29N
S56P
T94H

These experiments demonstrate that the stability of an antibody protein is enhanced by a factor of 100,000 when the above seven substitutions are made. However, there are alternative rosters of substitutions that would accomplish the same level of stability. Also, this level of stability improvement is not necessarily the upper limit that can be obtained.

The inventors have developed a unique database of sequences of ˜600 human antibody proteins, which provides additional resources for locating stabilizing amino acid changes. These sequences are disclosed in the following book chapter written by inventor: Stevens, F. J. et al. “Structural bases of light chain-related pathology”, The Antibodies Volume 5, pp 175-208 (Hardwood Academic Publishers, Australia, 1998), and incorporated herein by reference.

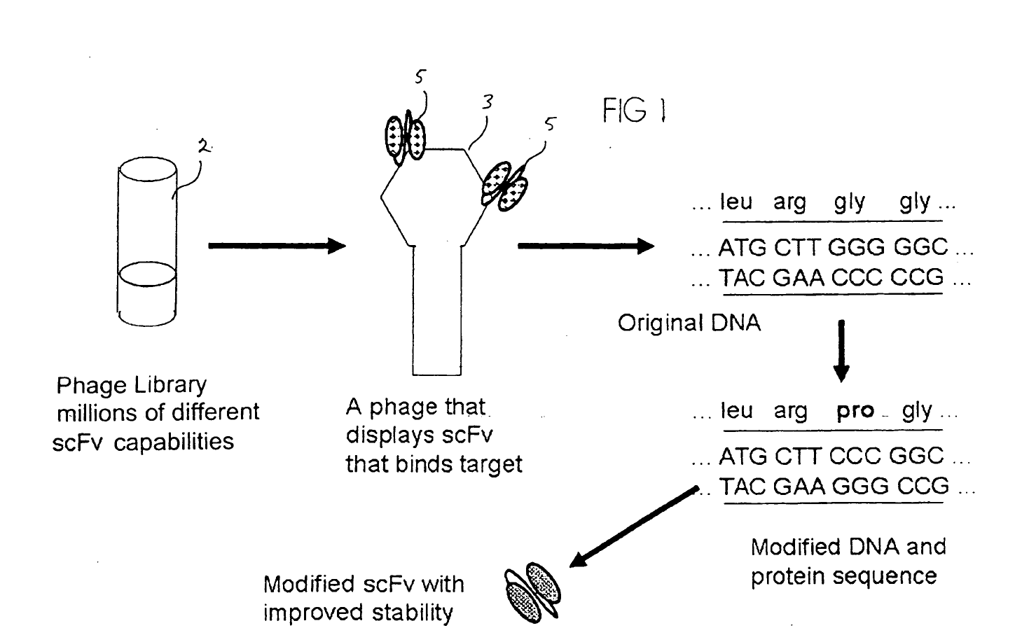

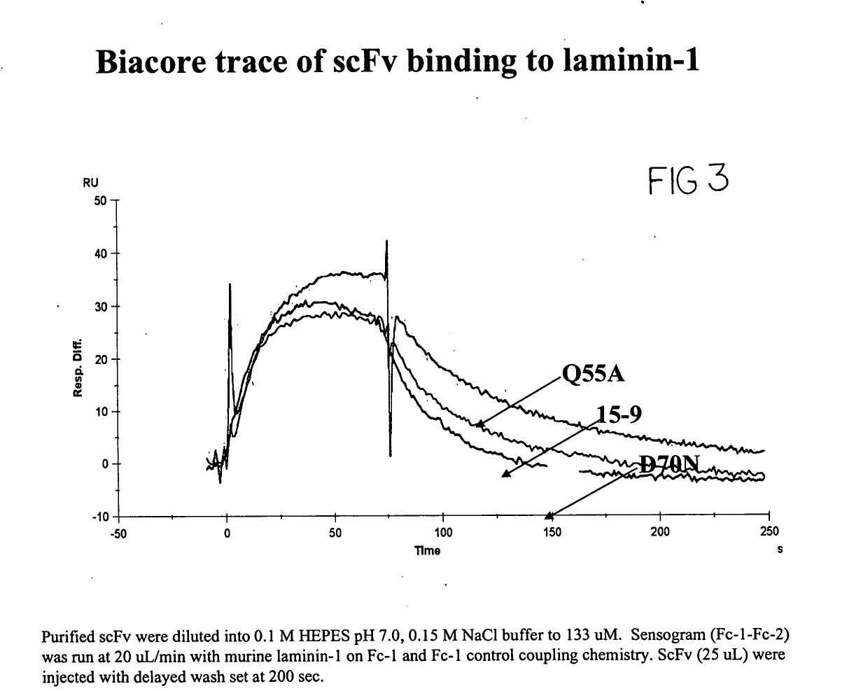

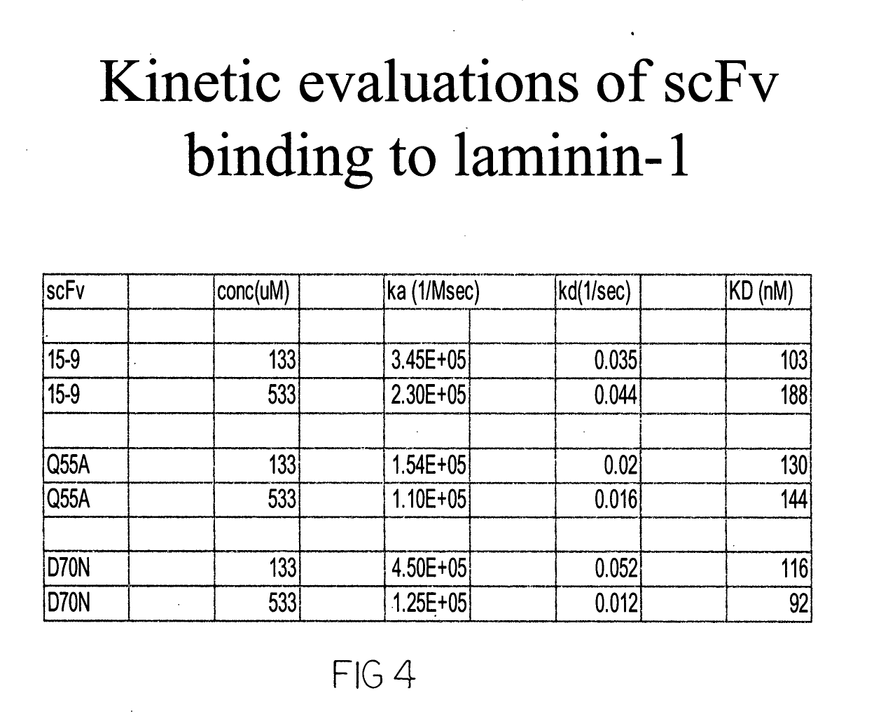

In operation, the protocol comprises determining antibody components that interact with a moiety associated with a particular disease state. FIG. 1 is a schematic representation of major steps in the process of developing a stable scFv (antibody) for binding to a pathogen or other target molecule such as laminin. Laminin is a large, noncollagenous glycoprotein with antigenic properties. It is localized in the basement membrane lamina lucida and functions to bind epithelial cells to the basement membrane. Evidence suggests that the protein plays a role in tumor invasion. The binding characteristics of mutated scFv to laminin are depicted in FIGS. 3 and 4. The scFv designation “15-9” in FIG. 4 is the name given to the variable fragment of kappa-1 as mutated by researchers at the University of Tennessee. RU designates “response unit.” The entire sequence of that fragment is enclosed herewith as the following SEQ. ID. No. 4:

(SEQ. ID. No. 4)

MKYLLPTAAAGLLLLAAQPAMAEVQLLESGGGLVQPGGSLRLSCAASGFT
FSSYAMSWVRQAPGKGLEWVSSIYTTGGYTGYADSVKGRFTISRDNSKNT
LYLQMNSLRAEDTAVYYCAKSTSSFDYWGQGTLVTVSSGGGGSGGGGSGG
GGSTDIQMTQSPSSLSASVGDRVTITCRASQSISSYLNWYQQKPGKAPKL
LIYGASSLQSGVPSRFSGSGSGTDFTLTISSLQPEDFATYYCQQTDYYPN
TFGQGTKVEIKRAAAHHHHHHGA

which depicts highest stability with only the following four mutations:
M4L
Q55A
S56P
D70N

Referring to FIG. 1, the first step is to select from a library 2 of phage that displays millions of scFv constructs, or portions thereof, having a wide variety of binding specificities. A small number 5 of at least portions of some of those constructs interact with the target. The scFv as displayed on the surface of the phage is effectively stable; i.e., because they are sequestered they can not aggregate/precipitate and sufficient numbers are functional, allowing the phage to bind to its target.

Suitable phage 3 are single stranded DNA viruses that infect a number of gram-negative bacteria. For example, filamentous phage particles, known as Ff, are suitable for display purposes. Exemplary specific phage include, but are not limited to phage lambda.

Most of the currently used phage display vectors use the N-terminus of pIII protein or pVIII protein to display the foreign peptide or protein. This protocol is disclosed in Smith G P and Scott J K (1993), Methods Enzymol 217:228-257 and incorporated herein by reference. Briefly, phage display protocols involve the introduction of exogenous peptide sequences into a location in the genome of the phage capsid proteins. The encoded peptides, for example representing scFv's or portions thereof, are expressed or otherwise displayed on the phage surface as a fusion product with one of the phage coat proteins. The genomes as altered in the instant invention are found in the amino acid sequence listing at the end of this specification. Exemplary phage display systems for use with the instant method of displaying stabilized antibody fragments are found in U.S. Pat. No. 7,041,441 and F. Yang, et al, Nat. Struct Biol (2000) 7(3), pp 230-7, both of which are incorporated herein by reference.

The small number of phage that bind target are amplified by infecting a bacterial culture; using very dilute phage suspensions assures that each culture is only infected with one phage, resulting in a homogeneous population of phage. The DNA of the scFv encoded by the phage is analyzed. Using site-specific mutagenesis, selected codons are modified to replace destabilizing amino acids and/or to introduce stabilizing amino acids. The modified DNA is introduced into an appropriate expression vector suitable for producing the protein in E. coli, yeast, or other production system. The scFv that results has the same binding characteristics as the original scFv that was extracted from the phage display library, but with substantially improved stability.

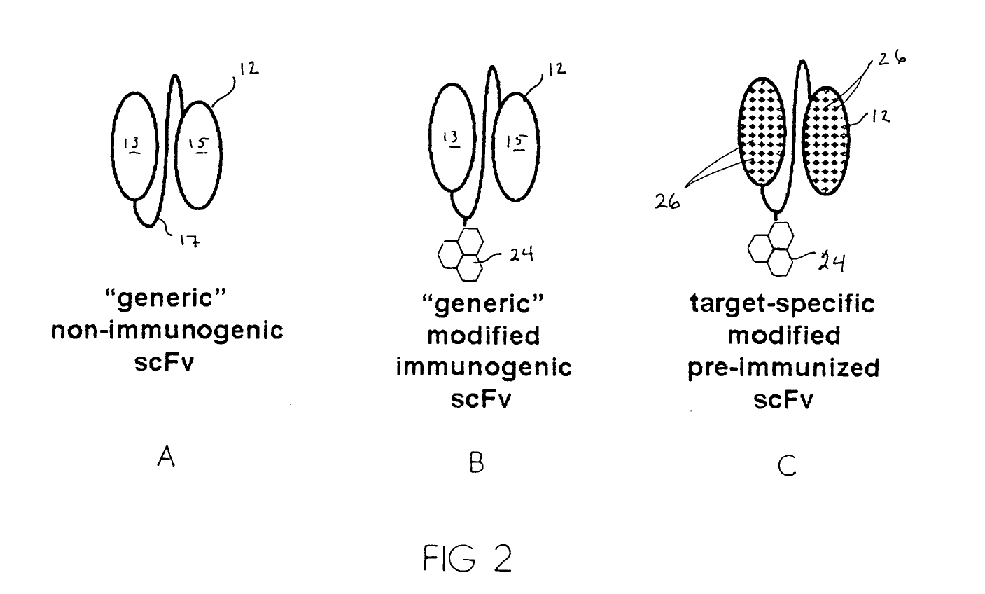

FIG. 2 is a schematic diagram of antibody component during various stages of its transformation to a universal vaccine particle, in accordance with an embodiment of the present invention. The natural scFv particle 12, is shown without any modification, that is to say, FIG. 2A depicts a generic non-immunogenic scFV, comprising a variable light chain 13 and a variable heavy chain 15. The variable components are linked via a peptide 17 containing a means for attaching a modifier molecule, discussed infra. Such means includes, but are not limited to amino acids (such as cysteine) containing sulphydryl groups.

In FIG. 2B, the modifier particle 24 is seen attached to the generic non-immunogenic scFV, thereby creating a “generic” modified immunogenic scFv. The construct depicted in FIG. 2B is the immunogen that is used for every scenario, in effect, a universal vaccine. The modifier particle, such as those listed in the heretofore referenced Livesay paper, induces a response from the body's immune system.

In FIG. 2C, the construct is complete in that a target-specific characteristic 26 is conferred to the particle. The checkered pattern on FIG. 2C indicates the antibody (element 5 in FIG. 1) used to target the pathogen, as determined by the phage library studies. The antibody was derived from the generic scFv initially used to immunize the individual and make that patient's system sensitive to the modifier 24. Antibody, or portions thereof target the pathogen in a myriad of ways. For example, a suitable binding motif of a targeting peptide (i.e. scFv) is a tripeptide motif appearing several times in different sequence contexts. As noted in Vendruscolo et al (2001), Nature 409, 641-645, and incorporated herein by reference, three amino-acid residues provide a suitable framework for structural formation and protein-protein interaction. However, inasmuch as suitable binding motifs are innumerable and no generalized rules exist in this technology, the three residue motif is provided merely as an illustration and not to limit the instant protocol.

Once the pathogen is contacted with the target-specific modified pre-immunized scFV, the patient's immune response, albeit generalized, attacks the pathogen.

In summary, the invented method for inducing a universal immune response in a patient comprises the following:
1. Obtaining human scFv having meta-specificity. In one embodiment of the invention, meta-specificity is defined as human scFv originally derived to bind to a target of non-human origin and therefore unlikely to interact with human tissues or molecules. For example, scFvs of mouse origin could be used as the immunogen, by-passing the need for hapten modification.
2. Modifying the peptide chain which connects the variable light (VL) and variable heavy (VH) antibody components of the scFv to create modified scFv. Modification includes covalently attaching a compound that induces an immune response. Such a compound includes those small molecules called haptens that evoke an immune response. Preferably, the compound does not resemble critical human metabolites or widely used (i.e. over the counter) medications. Generally, there is no limitation on the small molecules as long as a chemistry exists for their covalent attachment to the peptide linker.
3. Immunizing the patient with the modified scFv. This modified scFv is depicted as FIG. 2B.

In summarizing the initial inoculation steps, a patient is exposed to an immune response compound in a conventional vaccination protocol, such as is done for annual flu shots and traditional childhood vaccinations. In the instant protocol, the vaccine consists of metal specific human scFv modified by the immune inducing agent (i.e., hapten or haptens) of choice. In one embodiment of the protocol, the haptens are attached to proteins other than scFv in instances of concern about generating an autoimmune response to the human immune system. Alternatively an alternative scFv that is not reactive with human tissue is utilized. An scFv-hapten complex is preferred however as the immunogen would provide a more specific, higher affinity immune response to any pathogen-specific scFv-hapten used in the event of disease, thus the construct's role as a universal vaccine is realized. The scFv used as the hapten carrier is merely a proxy for the pathogen-specific scFv identified in phage studies, discussed immediately below. Once a pathogen presents itself in a population, the method continues as follows:
4. Obtaining protein coats for the pathogen, i.e., Virus X (VX).
5. Isolating and immobilizing VX coat proteins.
6. Introducing phage particles that “display” different antibodies on their outer surface. This step identifies which antibody components will interact with the pathogen.
7. Incubate and wash gently the phage library to remove non-binding, noninteracting constructs from the phage-VX mixture.
8. Wash more stringently to remove the phage particles that were bound so that those phage can be used to infect a bacterial culture.
9. Infect bacteria with those phage containing surface modifiers which interacted with VX proteins in step 6, and later isolated from ambiguous complexing events via washing steps 7 and 8. This infection step serves to amplify populations of the binders.
10. “Cloning” the phage to generate homogeneous populations, using standard cloning protocols found in the literature. These populations will provide DNA that encode scFv constructs that bind the coat proteins of the putative VX.
11. Modifying scFV-VX DNA to optimize stability of the protein it encodes.
12. Introducing scFv-VX DNA into bacteria or yeast.
13. Producing and purifying scFv-VX protein.
14. Modifying scFv-VX by attachment of the compound initially used to induce an immune response in step 2.
15. Infusing patients previously immunized with the modifying, immune response inducer.

Once patient infusion occurs, the scFv-VX-modifier particle, depicted in FIG. 2C, will bind to VX. The immune system will respond to the complex quickly in that antibodies will attach to the VX::scFv-VX-modifier particle complex. This induces the patient's white blood cells to remove the VX::scFv-VX-modifier particle complexes. The attachment of VX to the complex is a noncovalent interaction.

While the invention has been described in the foregoing with reference to details of the illustrated embodiments, these details are not intended to limit the scope of the invention as defined in the appended claims.

The embodiment of the invention in which an exclusive property or privilege is claimed is defined as follows:

1. A substance for detecting a target molecule, the substance comprising:
a) a first antibody molecule fragment exhibiting affinity for the target molecule; and
b) a second molecule in close spatial relationship to the fragment; the second molecule comprising a means to induce an immune response.

2. The substance as recited in claim 1 wherein the fragment is a single chain variable fragment that is manipulated to be more stable than the fragment in its native state.

3. The substance as recited in claim 1 wherein the fragment is a single chain variable fragment selected from mouse antibody, human antibody, and combinations thereof.

4. The substance as recited in claim 3 wherein the second molecule is a hapten, or peptide comprised of amino acids not found in biological systems, or a semi-conductor, or a metal, or synthetic material.

5. The substance as recited in claim 3 wherein the manipulation comprises replacement of an amino acid.

6. A method for treating a patient infected with a particular pathogen, the method comprising:
a) identifying antibody components which interact with a pathogen;
b) stabilizing the antibody components;
c) producing protein from the DNA;
d) modifying the protein by its attachment to a compound which induces an immune response in the patient; and
e) infusing patients with the modified protein.

7. The method as recited in claim 6 wherein the step of stabilizing the antibody components comprises:
a) infecting bacteria with phage containing the antibody components;
b) cloning the phage to generate homogeneous populations of DNA which encodes the antibody components; and
c) replacement of amino acid in the DNA such that the replacement of amino acid results in the antibody components being more stable than native antibody components.

8. The method as recited in claim 6 wherein the patients are previously immunized by a construct comprising the compound attached to scFv having meta-specificity.

9. The method as recited in claim 7 wherein the phage is a single-stranded DNA virus.

10. The method as recited in claim 7 wherein a single amino acid is replaced.

11. The method as recited in claim 7 wherein a plurality of amino acids are replaced.

12. The method as recited in claim 8 wherein the scFv is of human origin.

13. A universal antibody produced by the following process:
a) isolating a scFv component having meta-specificity;
b) producing quantities of the component; and
c) attaching a moiety to the produced scFv component quantities, whereby the moiety induces an immune response.

14. The universal antibody as recited in claim 13 wherein the scFv component does not induce an immune response by itself in a patient.

15. The universal antibody as recited in claim 13 wherein the step of producing quantities of the component further comprises:
d) integrating genetic sequences of the component into capsid DNA;
e) infecting bacteria with phage containing the capsid DNA; and
f) allowing the bacteria to multiply.

16. The universal antibody as recited in claim 13 wherein the moiety is contained in an immunogenic hapten.

17. The universal antibody as recited in claim 13 wherein the immune response is a human immune response.

18. The universal antibody as recited in claim 13 wherein in the immune response is a non-human immune response.

19. The universal antibody as recited in claim 13 wherein the scFv component is non-human in origin.


SEQUENCE LISTING (4 sequences)

SEQ ID NO: 1 - protein, 95 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLP

SEQ ID NO: 2 - protein, 245 aa, Artificial
  Example Sequence
MAAQIQLVQSGPELKKPGETVEISCKASGYTFTDYGMNWMKQAPGKSLKWMGWINTYTGEPTYADEFKGRFAFSLETSASTAYLQINNLKSEDMATYFCSRSMKGSYWGQGTLVTVSAGGGGSGGGGSGGGGSDVVMTQTPLTLSVTIGQPASISCKSSQSLLGSDGKTFLNWLLQRPGQSPKRLIYLVSKLDSGVPDRFTGSGSGTDFTLKISRVEAEDLGVYYCWQGTHLPQTFGGGTKLEIK

SEQ ID NO: 3 - protein, 245 aa, Artificial
  Example Sequence
MAAQIQLVQSGPELKKPGETVEISCKASGYTFTDYGMNWMKQAPGKSLKWMGWINTYTGEPTYADEFKGRFAFSLETSASTAYLQINNLKSEDMATYFCSRSMKGSYWGQGTLVTVSAGGGKSGGGGSGGGGSDVVMTQTPLTLSVTIGQPASISCKSSQSLLGSDGKTFLNWLLQRPGQSPKRLIYLVSKLDSGVPDRFTGSGSGTDFTLKISRVEAEDLGVYYCWQGTHLPQTFGGGTKLEIK

SEQ ID NO: 4 - protein, 273 aa, Artificial
  Example Sequence
MKYLLPTAAAGLLLLAAQPAMAEVQLLESGGGLVQPGGSLRLSCAASGFTFSSYAMSWVRQAPGKGLEWVSSIYTTGGYTGYADSVKGRFTISRDNSKNTLYLQMNSLRAEDTAVYYCAKSTSSFDYWGQGTLVTVSSGGGGSGGGGSGGGGSTDIQMTQSPSSLSASVGDRVTITCRASQSISSYLNWYQQKPGKAPKLLIYGASSLQSGVPSRFSGSGSGTDFTLTISSLQPEDFATYYCQQTDYYPNTFGQGTKVEIKRAAAHHHHHHGA